(19) United States
(12) Patent Application Publication
(10) Pub. No.: US 2006/0088488 A1
(43) Pub. Date: Apr. 27, 2006

(54) Dyeing composition comprising a mixture of at least one pigment and at least one electrophilic monomer

(76) Inventor: Gaelle Brun, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,321

(22) Filed: Oct. 13, 2005

Related U.S. Application Data
(60) Provisional application No. 60/646,494, filed on Jan. 25, 2005.

(30) Foreign Application Priority Data
Oct. 13, 2004 (FR) 0410802

(51) Int. Cl. A61K 8/00

(52) U.S. Cl. 424/70.1

(57) ABSTRACT

Disclosed is a dyeing keratin materials comprising applying to said materials at least one composition comprising, in a suitable dyeing medium, at least one electrophilic monomer, at least one colored pigment and at least one pigment that has a refractive index greater than the at least one colored pigment.

This application claims benefit of U.S. Provisional Application No. 60/646,494, filed Jan. 25, 2005, the contents of which are incorporated herein by reference. This application also claims benefit of priority under 35 U.S.C. § 119 to French Patent Application No. 04/10802, filed Oct. 13, 2004, the contents of which are also incorporated by reference.

The present disclosure relates to a composition for dyeing keratin materials, for example, keratin fibers such as the hair, comprising at least one mixture of certain pigments and at least one electrophilic monomer.

In the field of the dyeing of keratin fibers, it is already known practice to dye keratin fibers by various techniques using direct dyes or pigments for temporary colorations or using dye precursors for permanent colorations.

Temporary coloration or direct coloration comprises dyeing the keratin fibers with dye compositions comprising direct dyes. These dyes are colored and coloring molecules that have an affinity for keratin fibers. They are applied to the keratin fibers for a period of time necessary to obtain the desired coloration, and then rinsed off.

The conventional dyes that are used may be dyes of the nitrobenzene, anthraquinone, nitropyridine, azo, xanthene, acridine, azine or triarylmethane type or natural dyes.

Some of these dyes can be used under lightening conditions, which makes it possible to obtain visible colorations on dark hair.

It is also known practice to dye keratin fibers permanently by oxidation dyeing. This dyeing technique comprises applying to the keratin fibers a composition comprising dye precursors such as oxidation bases and couplers. These precursors, under the action of an oxidizing agent, will form at least one colored species in the hair.

The variety of molecules involved in oxidation bases and couplers makes it possible to obtain a rich array of colors and the colorations that result are generally permanent, strong, and/or resistant to outside agents, for example, to light, to bad weather, to washing, to perspiration and/or to rubbing.

In order to be visible on dark hair, these two dyeing techniques require prior or simultaneous bleaching of the keratin fibers. This bleaching step, carried out with an oxidizing agent such as hydrogen peroxide or persalts, may result in a not insignificant degradation of the keratin fibers, which may impair their cosmetic properties. The hair then has a tendency to become coarse, more difficult to disentangle and more brittle.

Another method of dyeing uses pigments. In fact, the use of a pigment at the surface of keratin fibers makes it possible in general to obtain visible colorations on dark hair since the surface pigment masks the natural color of the fiber. The use of a pigment for dyeing keratin fibers is, for example, described in French Patent Application No. FR 2 741 530, which recommends the use, for dyeing keratin fibers, of a composition comprising at least one dispersion of particles of a film-forming polymer comprising at least one acid function and at least one pigment dispersed in the continuous phase of said dispersion. The colorations obtained by this method of dyeing may have the drawback of having low shampoo fastness.

Compositions for treating the hair using compositions comprising electrophilic monomers are known from French Patent Application No. FR 2 833 489. Such a composition makes it possible to obtain hair that is completely coated and not greasy.

Thus it would be desirable to provide novel compositions for dyeing keratin materials, for example, keratin fibers such as the hair, which make it possible to obtain visible colorations on dark hair without it being necessary to lighten or bleach the fibers, and which exhibit good shampoo fastness.

Disclosed herein, therefore, is a dyeing composition comprising, in a suitable dyeing medium, at least one electrophilic monomer, at least one colored pigment and at least one pigment that has a refractive index greater than the colored pigment.

The composition in accordance with the present disclosure makes it possible to improve the visibility of the coloration on a dark keratin material. For example, in the case of dark keratin fibers, a visible coloration is obtained without it being necessary to lighten or bleach the keratin fibers and, consequently, without physical degradation of the keratin fibers. In addition, the combination of pigments of the composition disclosed herein makes it possible to obtain compositions that cover better, which makes it possible to more successfully mask white hairs.

In addition, the coloration disclosed herein may exhibit good resistance to the various attacking factors to which the hair may be subjected, such as shampoos, rubbing, light, bad weather, sweat and permanent reshaping operations. In at least one embodiment, the coloration shows good resistance with respect to shampoos.

Also disclosed herein is a process for dyeing keratin materials which comprises applying the composition disclosed herein to the keratin materials, and also the use of the composition for dyeing keratin materials, for example keratin fibers such as the hair.

Also disclosed herein is a kit comprising, firstly, a first composition comprising at least one pigment as defined above and, secondly, at least one electrophilic monomer.

As used herein, each pigment exhibits a water-solubility of less than 0.01% at 20° C., for example, less than 0.0001%, and exhibits an absorption ranging from 350 to 700 nm, for example, an absorption at one maximum.

The pigments that are useful herein may be in the form of pigmentary pastes and/or powders.

The at least one colored pigment may be chosen from organic pigments. As used herein, the term “organic pigment” means any pigment that corresponds to the definition in Ullmann's encyclopaedia in the organic pigment chapter. The organic pigment may, for example, be chosen from nitroso, nitro, azo, xanthene, quinoline, anthraquinone and phthalocyanin compounds, compounds of metal complex type, and isoindolinone, isoindoline, quinacridone, perinone, perylene, diketopyrrolopyrrole, thioindigo, dioxazine, triphenylmethane and quinophthalone compounds.

The organic pigment may be chosen, for example, from carmine, carbon black, aniline black, azo yellow, quinacridone, phthalocyanin blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified in the Color Index under the references CI 11725, 15510, 45370, 71105, the red pigments codified in the Color Index under the references CI 12085, CI 12120, 12370, 12420, 12490, 14700, 15525, 15580, 15620, 15630, 15800, 15850, 15865, 15880, 17200, 26100, 45380, 45410, 58000, 73360, 73915, 75470, and the pigments obtained by oxidative polymerization of indole or phenolic derivatives as described in French Patent No. FR 2 679 771.

These organic pigments may also be in the form of composite pigments as described in European Patent No. EP 1 184 426. This composite pigment may be composed, for example, of at least one particle containing an inorganic core, at least one binder providing attachment of the organic pigments to the core, and at least one organic pigment that at least partially covers the core.

By way of example, non-limiting mention may be made of pigmentary pastes of organic pigment such as the products sold by the company Hoechst under the name:

JAUNE COSMENYL IOG: Pigment YELLOW 3 (CI 11710)

JAUNE COSMENYL G: Pigment YELLOW 1 (CI 11680)

ORANGE COSMENYL GR: Pigment ORANGE 43 (CI 71105)

ROUGE COSMENYL R″: Pigment RED 4 (CI 12085)

CARMIN COSMENYL FB: Pigment RED 5 (CI 12490)

VIOLET COSMENYL RL: Pigment VIOLET 23 (CI 51319)

BLEU COSMENYL A2R: Pigment BLUE 15.1 (CI 74160)

VERT COSMENYL GG: Pigment GREEN 7 (CI 74260)

NOIR COSMENYL R Pigment BLACK 7 (CI 77266)

The at least one colored pigment may also be chosen from lakes. As used herein, the term “lake” means dyes adsorbed onto insoluble particles, the assembly thus obtained remaining insoluble during use. The inorganic substrates onto which the dyes are adsorbed are, for example, alumina, silica, calcium sodium borosilicate or calcium aluminium borosilicate, and aluminium. Among the organic dyes, non-limiting mention may be made of cochineal carmine.

By way of examples of lakes, non-limiting mention may be made of the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 1 O (CI 77 002), D & C Green 3 (CI 42 053), D & C Blue 1 (CI 42 090).

The at least one colored pigment may also be a pigment with special effects. As used herein, the term “pigment with special effects” means pigments that generally create a colored appearance (characterized by a certain shade, a certain vivacity and a certain level of luminance) that is non-uniform and that changes as a function of the conditions of observation (light, temperature, angles of observation, etc.). Such pigments are consequently in contrast to white or colored pigments, which provide a standard opaque, semi-transparent or transparent uniform shade.

Two types of pigments with special effects exist: those with a low refractive index, such as fluorescent, photochromic or thermochromic pigments, and those with a high refractive index, such as pearlescent agents or flakes.

By way of pigments with special effects, non-limiting mention may be made of pearlescent pigments such as white pearlescent pigments, for instance mica coated with titanium or mica coated with bismuth oxychloride, colored pearlescent pigments such as mica coated with titanium and with iron oxides, mica coated with titanium and with ferric blue or with chromium oxide, mica coated with titanium and with an organic pigment of the abovementioned type, and also pearlescent pigments based on bismuth oxychloride.

The pigments with special effects may also comprise fluorescent pigments, whether they are substances that are fluorescent in daylight or that produce ultraviolet fluorescence, phosphorescent pigments, photochromic pigments, and/or thermochromic pigments.

The composition as disclosed herein comprises at least one pigment that has a refractive index greater than the colored pigment.

The at least one pigment having a refractive index higher than the at least one colored pigment may be chosen from mineral pigments. As used herein, the term “mineral pigment” means any pigment that corresponds to the definition in Ullmann's encyclopaedia in the inorganic pigment chapter. Among mineral pigments useful herein, non-limiting mention may be made of zirconium oxides or cerium oxides, and also iron oxides or chromium oxides, manganese violet, ultramarine blue, chromium hydrate, ferric blue and titanium dioxide. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The at least one pigment having a refractive index higher than the at least one colored pigment may also be chosen from pearlescent pigments such as white pearlescent pigments, for example mica coated with titanium or with bismuth oxychloride, colored pearlescent pigments such mica coated with titanium and with iron oxides, mica coated with titanium and with ferric blue or with chromium oxide, mica coated with titanium and with an organic pigment as defined above, and also pearlescent pigments based on bismuth oxychloride. By way of example, non-limiting mention may be made of the pigments CELLINI sold by the company Engelhard (Mica-TiO₂-lake), PRESTIGE sold by the company Eckart (Mica-TiO₂), or COLORONA sold by Merck (Mica-TiO₂—Fe₂O₃).

In addition to the pearlescent agents on a mica support, multilayer pigments based on synthetic substrates such as alumina, silica, calcium sodium borosilicate or calcium aluminium borosilicate, and aluminium may also be used in some embodiments.

The at least one colored pigment and at least one pigment having a refractive index higher than the at least one colored pigment each range in size from 10 nm to 200 μm, for example, from 20 nm to 80 μm, and further, for example, from 30 nm to 50 μm.

The at least one colored pigment and at least one pigment having a refractive index higher than the at least one colored pigment are each generally present in the composition in amounts ranging from 0.05% to 50%, for example, from 0.1 to 35%, by weight, relative to the total weight of the composition,

The refractive index of the pigments can be measured according to known methods for measuring these indices. In the case of the organic pigments, the refractive index can be measured according to the method described in the article “The refractive index of organic pigments. Its determination and significance”, by A. C. Cooper, B.SC., A.R.I.C., in Journal of the Oil & Colour Chemists' Association, September 1948, No. 339, Vol. XXXI.

The at least one electrophilic monomer in the presently disclosed composition may be chosen from, for example,
the benzylidenemalononitrile derivatives (A), 2-(4-chlorobenzylidene)malononitrile (A1), ethyl 2-cyano-3-phenylacrylate (B), ethyl 2-cyano-3-(4-chlorophenyl)acrylate (B1) described in Sayyah, J. Polymer Research, 2000, p 97:

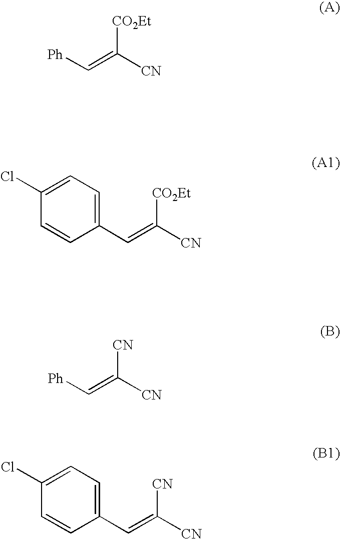

Methylidenemalonate derivates such as:
The diethyl 2-methylenemalonate (C) by Hopff, Makromoleculare Chemie, 1961, p 95, De Keyser, J. Pharm. Sci, 1991, p 67, and Klemarczyk, Polymer, 1998, p 173: 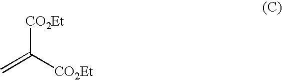
the ethyl 2-ethoxycarbonylmethyleneoxycarbonylacrylate (D) by Breton, Biomaterials, 1998, p 271 and Couvreur, Pharmaceutical Research, 1994, p 1270.

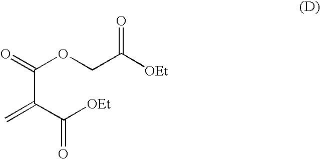

Itaconate and itaconimide derivatives such as:
the dimethyl itaconate (E) by Bachrach, European Polymer Journal, 1976, p 563: 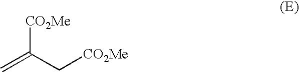
N-butylitaconimide (F), N-(4-tolyl)itaconimide (G), N-(2-ethylphenyl)itaconimide (H), N-(2,6-diethylphenyl)itaconimide (I) by Wanatabe, J. Polymer Science: Part A: Polymer chemistry, 1994, p 2073: 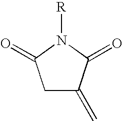
R═Bu (F), 4-tolyl (G), 2-ethylphenyl (H), 2,6-diethylphenyl (I).

The methyl α-(methylsulphonyl)acrylate (K), ethyl α-(methylsulphonyl)acrylate (L), methyl α-(tert-butylsulphonyl)acrylate (M), tert-butyl α-(methylsulphonyl)acrylate (N) and tert-butyl α-(tert-butylsulphonyl)acrylate (O) derivatives by Gipstein, J. Org. Chem. 1980, p 1486 and the 1,1-bis(methylsulphonyl)ethylene (P), 1-acetyl-1-methylsulphonylethylene (Q), methyl α-(methylsulphonyl)vinylsulphonate (R), and α-methylsulphonylacrylonitrile (S) derivatives described by Shearer, U.S. Pat. No. 2,748,050. 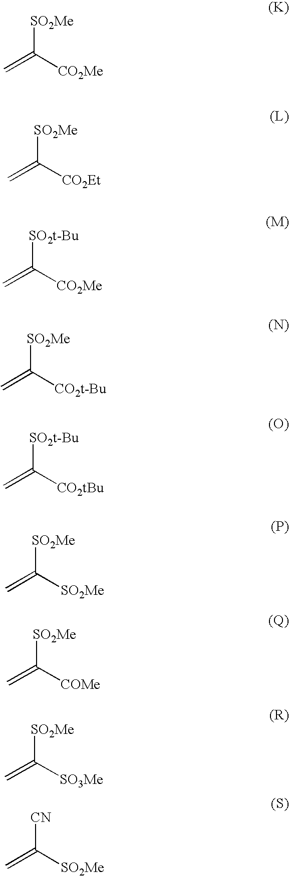
The methyl vinyl sulphone (T) and phenyl vinyl sulphone (U) derivatives by Boor, J. Polymer Science, 1971, p 249:

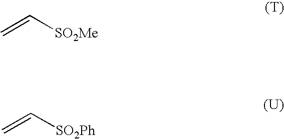

The phenyl vinyl sulphoxide derivative (V) by Kanga, Polymer preprints (ACS, Division of Polymer Chemistry), 1987, p 322:

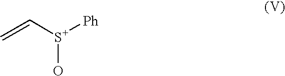

The 3-methyl-N-(phenylsulphonyl)-1-aza-1,3-butadiene derivative (W) by Bonner, Polymer Bulletin, 1992, p 517:

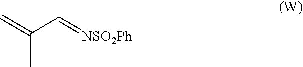

Acrylate and acrylamide derivatives such as:
N-propyl-N-(3-triisopropoxysilylpropyl)acrylamide (X) and N-propyl-N-(3-triethoxysilylpropyl)acrylamide (Y) by Kobayashi, Journal of Polymer Science, Part A: Polymer Chemistry, 2005, p 2754: 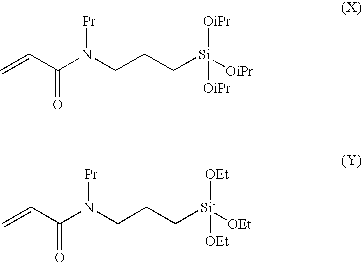
2-hydroxyethyl acrylate (Z) and 2-hydroxyethyl methacrylate (AA) by Rozenberg, International Journal of Plastics Technology, 2003, p 17: 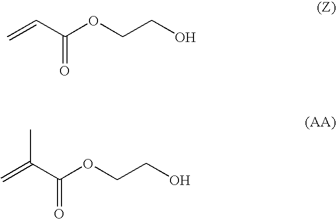
N-butyl acrylate (AB) by Schmitt, Macromolecules, 2001, p 2115, and
tert-butyl acrylate (AC) by Ishizone, Macromolecules, 1999, p 955.

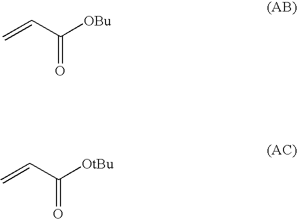

The at least one electrophilic monomer useful herein may be cyclic or linear. When it is cyclic, the electrophilic group may be exocyclic, i.e., it is not an integral part of the cyclic structure of the monomer.

According to one embodiment, these monomers have at least two electrophilic groups.

By way of example of monomers having at least two electrophilic groups, non-limiting mention may be made of the monomers of formula (A): 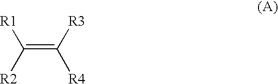
wherein:

R1 and R2, which may be identical or different, are each chosen from groups with little or no electron-withdrawing effect (with little or no inductive withdrawing effect) such as:
a hydrogen atom,
saturated or unsaturated, linear, branched or cyclic hydrocarbon-based groups, for example, comprising from 1 to 20 carbons, further, for example, from 1 to 10, carbon atoms, and optionally comprising at least one atom chosen from nitrogen, oxygen and sulphur atoms, and optionally substituted with at least one group chosen from —OR, —COOR, —COR, —SH, —SR, —OH, and halogen atoms,
modified or unmodified polyorganosiloxane residues,
polyoxyalkylene groups,

R3 and R4, which may be identical or different, are each chosen from electron-withdrawing groups (or groups with inductive withdrawing effect, i.e., electrophilic), for example, chosen from the groups —N(R)₃+, —S(R)₂+, —SH2+, —NH3+, —NO₂, —SO₂R, —C═N, —COOH, —COOR, —COSR, —CONH₂, —CONHR, —F, —Cl, —Br, —I, —OR, —COR, —SH, —SR and —OH, linear or branched alkenyl groups, linear or branched alkynyl groups, mono- or polyfluoro(C₁-C₄)alkyl groups, aryl groups such as phenyl, and aryloxy groups such as phenoxy,

wherein R denotes a saturated or unsaturated, linear, branched or cyclic hydrocarbon-based group, for example, comprising from 1 to 20, further, for example, from 1 to 10, carbon atoms, and optionally comprising at least one atom chosen from nitrogen, oxygen and sulphur atoms, and optionally substituted with at least one group chosen from —OR′, —COOR′, —COR′, —SH, —SR′, —OH, halogen atoms, or a residue of a polymer that can be obtained by radical polymerization, by polycondensation or by ring opening, R′ denoting a C₁-C₁₀ alkyl group.

As used herein, the term “electron-withdrawing group, or group with inductive withdrawing effect (—I)” means any group that is more electronegative than carbon. Reference may be made to the work PR Wells Prog. Phys. Org. Chem., Vol. 6, 111 (1968).

As used herein, the term “group with little or no electron-withdrawing effect” means any group whose electronegativity is less than or equal to that of carbon.

The alkenyl or alkynyl groups may, for example, have 2 to 20 carbon atoms, futher, for example, from 2 to 10 carbon atoms.

The saturated or unsaturated, linear, branched or cyclic hydrocarbon-based groups, for example, comprising from 1 to 20 carbon atoms, may be chosen from linear or branched alkyl, alkenyl or alkynyl groups, such as methyl, ethyl, n-butyl, tert-butyl, isobutyl, pentyl, hexyl, octyl, butenyl or butynyl; cycloalkyl groups and aromatic groups.

The substituted hydrocarbon-based group may, for example, be chosen from hydroxyalkyl and polyhaloalkyl groups.

The unmodified polyorganosiloxanes may be chosen from, for example, polyalkylsiloxanes such as polydimethylsiloxanes, polyarylsiloxanes such as polyphenylsiloxanes, and polyarylalkylsiloxanes such as polymethylphenylsiloxanes.

The modified polyorganosiloxanes may be chosen from, for example, polydimethylsiloxanes comprising polyoxyalkylene and/or siloxy and/or silanol and/or amine and/or imine and/or fluoroalkyl groups.

The polyoxyalkylene groups may be chosen from, for instance, polyoxyethylene groups and polyoxypropylene groups, having, for example, from 1 to 200 oxyalkylenated units.

Among mono- or polyfluoroalkyl groups, mention may be made of groups such as —(CH₂)ₙ—(CF₂)ₘ—CF₃ or —(CH₂)ₙ—(CF₂)ₘ—CHF₂ wherein n ranges from 1 to 20 and m ranges from 1 to 20.

The substituents R1 to R4 may optionally be substituted with a group having a cosmetic activity. The cosmetic activities may, for example, be obtained from groups with coloring, antioxidant, UV-screening and conditioning functions.

By way of examples of a group with a coloring function, mention may be made of azo, quinone, methine, cyanomethine and triarylmethane groups.

By way of examples of a group with an antioxidant function, mention may made of groups of butylhydroxyanisole (BHA), butylhydroxytoluene (BHT) and vitamin E type.

By way of examples of a group with a UV-screening function, mention may be made of groups of benzophenone, cinnamate, benzoate, benzylidenecamphor and dibenzoylmethane types.

By way of examples of a group with a conditioning function, mention may be made of cationic and fatty ester-type groups.

In at least one embodiment, the monomers mentioned above may be chosen from monomers of the cyanoacrylate family and derivatives thereof of formula (B):

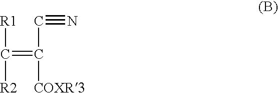

wherein:
X is chosen from NH, S and O,
R1 and R2 have the same meanings as above, and in at least one embodiment, R1 and R2 are each a hydrogen atom,
R′3 is chosen from a hydrogen atom and R as defined in formula (A).

In at least one embodiment, X is O.

As compounds of formula (B), mention may be made of the monomers:

a) belonging to the family of polyfluoroalkyl 2-cyanoacrylates, such as: 2-cyano-2-propenoic acid 2,2,3,3-tetrafluoropropyl ester of formula: 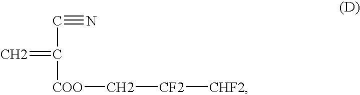
or else 2-cyano-2-propenoic acid 2,2,2-trifluoroethyl ester of formula:

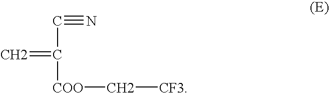

b) alkyl or alkoxyalkyl 2-cyanoacrylates

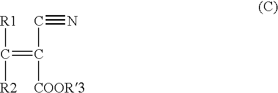

wherein R′3 is chosen from C₁-C₁₀ alkyl and (C₁-C₄)alkoxy(C₁-C₁₀)alkyl radicals.

Mention may be made, for use in at least one embodiment, of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and isoamyl cyanoacrylate.

In at least one embodiment, the monomer is chosen from monomers b). According to another embodiment, the cyanoacrylate monomers may be chosen from C₆-C₁₀ alkyl cyanoacrylates.

In at least one embodiment, the monomers may be chosen from the octyl cyanoacrylates of formula (F) and mixtures thereof: 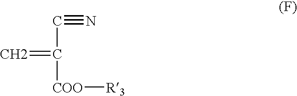
wherein: R′₃ is chosen from —(CH₂)₇—CH₃, —CH(CH₃)—(CH₂)₅—CH₃, —CH₂—CH(C₂H₅)—(CH₂)₃—CH₃, —(CH₂)₅—CH(CH₃)—CH₃, and —(CH₂)₄—CH(C₂H₅)—CH₃.

In one embodiment, the monomers may be covalently attached to supports such as polymers, oligomers or dendrimers. The polymer or the oligomer may be linear, branched, in the form of a comb or in the form of a block. The distribution of the monomers disclosed herein on the polymeric, oligomeric or dendritic structure may be random, at the end position or in the form of blocks.

In the composition of the present disclosure, the cyanoacrylate monomer is present in an amount ranging from 0.1 to 80% by weight, relative to the total weight of the composition, for example, from 1 to 50%.

As disclosed herein, the at least one electrophilic monomer is a monomer capable of polymerizing via the anionic pathway in the presence of a nucleophilic agent. As used herein, the term “anionic polymerization” means the mechanism defined in the work “Advanced Organic Chemistry”, Third Edition by Jerry March, pages 151 to 161.

The nucleophilic agents capable of initiating anionic polymerization are systems known in themselves, capable of generating a carbanion on contact with a nucleophilic agent, such as the hydroxide ions contained in water at neutral pH. As used herein, the term “carbanion” means the chemical species defined in “Advanced Organic Chemistry”, Third Edition, by Jerry March, page 141.

The nucleophilic agents may be applied independently of the composition disclosed herein. The nucleophilic agent may also be added to the composition disclosed herein at the time of use.

The nucleophilic agent is chosen from a molecular compound, an oligomer, a dendrimer or a polymer having nucleophilic functions. Without implied limitation, nucleophilic functions that may be mentioned include the functions: R₂N⁻, NH₂⁻, Ph₃C⁻, R₃C⁻, PhNH⁻, pyridine, ArS⁻, R—C⁻C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, wherein Ph is a phenyl group; Ar is an aryl group and R is a C₁-C₁₀ alkyl group.

The at least one electrophilic monomer can be synthesized according to the known methods described in the art. For example, the cyanoacrylate monomers can be synthesized according to the teaching of U.S. Pat. No. 3,527,224, U.S. Pat. No. 3,591,767, U.S. Pat. No. 3,667,472, U.S. Pat. No. 3,995,641, U.S. Pat. No. 4,035,334 and U.S. Pat. No. 4,650,826.

The appropriate dyeing medium used in the composition disclosed herein may be, for example, an anhydrous and non-hygroscopic medium. As used herein, the term “anhydrous medium” means a medium containing less than 1% of water.

According to at least one embodiment, the dyeing medium of the composition disclosed herein may be chosen from:
aromatic alcohols such as benzyl alcohol;
fatty alcohols;
modified or unmodified polyols, such as glycerol, glycol, propylene glycol, dipropylene glycol, butylene glycol or butyl diglycol;
volatile or non-volatile silicones, such as cyclopentasiloxane, cyclohexasiloxane, polydimethylsiloxanes that are unmodified or modified with phenyl and/or siloxy and/or silanol and/or amine and/or imine and/or fluoroalkyl and/or carboxylic and/or betaine and/or quaternary ammonium, etc., functions;
mineral, organic or plant oils;
oxyethylenated or non-ethoxyethylenated waxes, paraffins, alkanes, for example, C₅ to C₁₀ alkanes; and
fatty acids, fatty amides, fatty esters, for example, fatty alcohol benzoates or salicylates.

According to another embodiment, the medium may comprise silicones such as polydimethylsiloxanes and modified polydimethylsiloxanes.

For example, the organic compounds may be chosen from compounds that are liquid at a temperature of 25° C. and under 105 Pa (760 mmHg).

The dyeing medium of the presently disclosed composition may also be in the form of an emulsion and/or may be encapsulated, wherein the at least one electrophilic monomer is maintained in an anhydrous medium until the moment of use. When the dyeing medium is an emulsion, this emulsion comprises, for example, a dispersed or continuous phase that may comprise water, C₁-C₄ aliphatic alcohols or mixtures thereof, and an anhydrous organic phase comprising the monomer. In the case of capsules or microcapsules, the capsule may contain the monomer in an anhydrous medium and may be dispersed in an anhydrous medium as defined above, water, C₁-C₄ aliphatic alcohols, or mixtures thereof.

Polymerization inhibitors, for example, anionic and/or radical polymerization inhibitors, may also be introduced into the composition disclosed herein in order to increase the stability of the composition over time. Without implied limitation, mention may be made of the following polymerization inhibitors: sulphur dioxide, nitric oxide, organic acids such as a sulphonic acid or phosphoric acid, acetic acid, lactone, boron trifluoride, hydroquinone and its derivatives, such as hydroquinone monoethyl ether or tert-butyl hydroquinone, benzoquinone and its derivatives, such as duroquinone, catechol and its derivatives, such as t-butyl catechol and methoxycatechol, anisole and its derivatives, such as methoxy-anisole or hydroxyanisole, pyrogallol and its derivatives, p-methoxyphenol, hydroxybutyltoluene, alkyl sulphates, alkyl sulphites, alkyl sulphones, alkyl sulphoxides, alkyl sulphides, mercaptans, and mixtures thereof. In at least one embodiment, the alkyl groups may comprise groups having 1 to 6 carbon atoms.

The concentration of inhibitor in the composition disclosed herein may range from 10 ppm to 10%, for example, from 50 ppm to 5% by weight.

The composition disclosed herein may also comprise at least one polymer that does not exhibit any reactivity on the electrophilic monomers and are capable of increasing the viscosity of the composition. The increase in viscosity makes it possible to reduce the rate of polymerization of the electrophilic monomers. To do this, it is possible to add to the composition disclosed herein, and in a nonexhaustive manner, poly(methyl methacrylate) (PMMA) or else the copolymers based on cyanoacrylate as described in U.S. Pat. No. 6,224,622.

The composition disclosed herein may also comprise at least one filler. The at least one filler may be chosen from, without implied limitation, lamellar or non-lamellar, mineral or synthetic, and colorless or white particles. They may be present in an amount ranging from 0 to 48% by weight, relative to the total weight of the composition, for example, from 0.01 to 30% by weight, and further, for example, from 0.02% to 20% by weight. Mention may be made, for example, of talc, zinc stearate, mica, kaolin, polyamide powders (Nylon@) (Orgasol from Atochem), polyethylene powders, tetrafluoroethylene polymer powders (TeflonB), starch, boron nitride, polymeric microspheres such as those made of polyvinylidene chloride/acrylonitrile, for instance Expancel (Nobel Industrie), or made of acrylic acid copolymers (PolytrapB from the company Dow Corning), and microbeads of silicone resin (TospearlsO from Toshiba, for example), elastomeric organopolysiloxanes.

The composition disclosed herein may also comprise at least one conventional pigment. It may also comprise metal powders or particles, such as aluminium, zinc, copper, etc., powders or particles.

The composition may also comprise at least one conventional cosmetic active agent. Mention may be made, without implied limitation, of reducing agents, oxidizing agents, fatty substances, silicones, thickeners, softening agents, anti-foaming agents, moisturizers, emollients, basifying agents, elastomers, plasticizers, sunscreens, direct or oxidation dyes, clays, colloidal minerals, fragrances, peptizers, preserving agents, anionic, cationic, amphoteric, zwitterionic or non-ionic surfactants, fixing or non-fixing polymers, conditioning polymers, proteins, vitamins, etc.

These compositions may be in various forms, such as lotions, sprays or mousses, and may be applied in the form of a shampoo or of a conditioner.

In the case of sprays, the composition disclosed herein may comprise a propellant. The propellant comprises the compressed or liquefied gases usually employed for the preparation of aerosol compositions. Use may be made of air, carbon dioxide, compressed nitrogen, or else a soluble gas such as dimethyl ether, non-halogenated hydrocarbons or halogenated (for example, fluorinated) hydrocarbons, and mixtures thereof.

Also disclosed herein is a process whereby the composition disclosed herein is applied to keratin materials, for example, keratin fibers such as the hair, in the presence of a nucleophilic agent.

According to at least one embodiment of this process, the nucleophilic agent capable of initiating the polymerization of the cyanoacrylate monomer may be applied beforehand to the keratin fibers. The nucleophilic agent may be used pure, in solution, or in the form of an emulsion, or may be encapsulated. It may also be added to the anhydrous composition at the time of use, just before application to the keratin fibers.

For example, this nucleophilic agent may be water. This water may be provided, for example, by prior moistening of the keratin fibers. It may also be added directly to the composition before application.

According to at least one embodiment, it is possible to modulate the polymerization kinetics by moistening the fiber beforehand with an aqueous solution, the pH of which has been adjusted using a base, an acid or an acid/base mixture. The acid and/or the base may be inorganic or organic.

According to another embodiment, the process disclosed herein may be carried out in several steps: a first step which comprises applying a composition comprising the at least one colored pigment and at least one pigment that has a refractive index greater than the at least one colored pigment to the fibers, and a second step comprising applying a composition comprising the at least one electrophilic monomer, wherein the nucleophilic agent is present in the composition comprising the pigment or in a separate composition.

For example, the composition comprising the at least one colored pigment and at least one pigment that has a refractive index greater than the at least one colored pigment may be an aqueous solution of pigments, which makes it possible to moisten the fiber and to initiate the polymerization when the cyanoacrylate monomer is applied.

One embodiment of the process disclosed herein comprises applying either the at least one electrophilic monomer and the at least one colored pigment and at least one pigment that has a refractive index greater than the at least one colored pigment using the same composition, or in applying firstly the at least one colored pigment and at least one pigment that has a refractive index greater than the at least one colored pigment, and then the at least one electrophilic monomer.

In another embodiment, the process disclosed herein may comprise additional intermediate or final steps, such as the application of a cosmetic product, a rinsing step or a drying step. The drying can be carried out with a hood, with a hairdryer and/or with straightening tongs. For example, the application of the compositions disclosed herein may be followed by rinsing.

It is also possible to perform multiple applications of the composition disclosed herein in order to obtain a superposition of layers so as to attain specific properties of the deposit in terms of chemical nature, mechanical strength, thickness, appearance, feel.

In order to improve, inter alia, the adhesion of the poly(cyanoacrylate) formed in situ, the fiber may be pretreated with polymers of all types.

In order to modulate the anionic polymerization kinetics, the nucleophilicity of the fiber can also be increased by chemical conversion of the keratin fibers. By way of example, non-limiting mention may be made of reduction of the disulphide bridges, that partly make up keratin, to thiols before application of the composition disclosed herein. In a nonexhaustive manner, mention may be made, as agents that reduce the disulphide bridges partly making up keratin, of the following compounds: anhydrous sodium thiosulphate, powdered sodium metabisulphite, thiourea, ammonium sulphite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulphoxylate, isooctyl thioglycolate, dl-cysteine, monoethanolamine thioglycolate.

The application of a composition disclosed herein may also be preceded by a hair treatment such as direct or oxidation dyeing.

In one embodiment, the monomers may be chosen from monomers capable of polymerizing on keratin fibers under cosmetically acceptable conditions. For example, the polymerization of the monomer may be carried out at a temperature of less than or equal to 80° C., which does not prevent the application from being finished with drying under a hood, blow-drying, or the use of a flat heating iron or curling tongs.

Also disclosed herein is a dyeing kit comprising a first composition comprsising at least one pigment and a second composition which comprising the at least one electrophilic monomer and, optionally, a third composition which comprising the at least one nucleophilic agent. According to this embodiment, the composition comprising the at least one pigment is an aqueous composition and the composition comprising the at least one electrophilic monomer is an anhydrous composition.

According to another embodiment, the kit comprises a first anhydrous composition which comprises the at least one pigment and the at least one electrophilic monomer, and a second composition which comprises at least one nucleophilic agent.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

### EXAMPLES

Trials were carried out using the following compounds:

Monomer: 2-octyl 2-cyanoacrylate stabilized with 1% of phosphoric acid, known as RITE LOK CON895, sold by the company Chemence.

Pigment 1: Disperse Red 122, CI 73915, sold by Sun under the name Sunfast Magenta 122, which has a refractive index of less than 2.75.

Pigment 2: Untreated titanium oxide sold by WCD under the name Titanium Dioxyde USP BC 2559, which has a refractive index of 2.75.

Dyeing Medium:

50% of alpha, omega-dihydroxy polydimethyl-siloxane/cyclopentadimethylsiloxane mixture (14.7/85.3) sold by Dow Corning under the name DC 1501 Fluid,

50% of cyclopentadimethylsiloxane sold by Dow Corning under the name DC 245 Fluid.

### Example 1

An aqueous composition was prepared with 10% of pigment 1 and 10% of pigment 2. 0.5 g of this aqueous solution was applied to 1 g of a lock of clean and dry natural hair with a tone strength equal to 4, which corresponds to a natural chestnut shade according to the classification of natural shades described in “Science des Traitements Capillaires” [The science of hair treatments] by C. Zviak, published by Masson 1988, p. 278.

The lock was subsequently dried under a hood, and then moistened with 0.5 g of water. 0.5 g of a composition comprising the dyeing medium described above and 10% by weight of cyanoacrylate monomers was then applied to this moistened lock.

After an application time of 10 minutes, the lock was dried for 2 minutes with a hairdryer.

The lock obtained was colored pink and the color obtained persisted after at least six shampooing operations. In addition, the color obtained was more visible than that obtained using a composition comprising only pigment 1.

### Example 2

A composition comprising 10% of pigment 1 and 10% of pigment 2 in the dyeing medium described above was prepared. The cyanoacrylate monomer was added to this composition so as to obtain a final concentration of 10% by weight of monomers. 0.5 g of this composition was applied to a lock of clean and dry natural hair with a tone strength equal to 4, moistened with 0.5 g of water.

After an application time of 10 minutes, the lock was dried for 2 minutes with a hairdryer. A pink-colored lock was obtained as above.

The coloration thus obtained was extremely shampoo-fast.

### Examples 3 to 10

The following compositions were prepared for the production of a dyeing composition.

Colored Composition A

| | |
|---|---|
| Pigment Red 122, | 46 g |
| sold under the name Sunfast Magenta 122228-2410 by Sun | |
| (refractive index less than 2.75) | |
| Titanium dioxide USP BC 2559 from Kronos International | 46 g |
| (refractive index less than 2.75) | |
| Polyhydroxystearic acid dispersant sold under the name | 8 g |
| Octacare DSP OL 300 by Avecia | |

Colored Composition L

| | |
|---|---|
| Red-orange fluorescent organic pigment sold under the name | 46 g |
| Sunbrite SG2516 Red Orange by the company Sun | |
| (refractive index less than 1.64) | |
| Yellow iron oxide sold under the name Sunpuro Yellow C33-9001 | 46 g |
| by the company Sun | |
| (refractive index less than 2.9) | |
| Polyhydroxystearic acid dispersant sold under the name | 8 g |
| Octacare DSP OL 300 by Avecia | |

### Example 3

### Methylheptyl Cyanoacrylate Monomer

The following composition was prepared:

| | |
|---|---|
| DC 1501 Fluid | 40 g |
| DC 245 Fluid | 40 g |
| A composition A | 10 g |
| Methylheptyl cyanoacrylate from Chemence | 10 g |

1.5 g of the composition was applied to a lock of 1 g of clean and moist chestnut hair. After a period of 15 minutes, the lock was dried with a hairdryer for 2 minutes.

The lock was colored and the coloration obtained was shampoo-fast. The coloration obtained was more visible than if a composition comprising only the pigment with a low refractive index had been applied.

### Example 4

### Methylheptyl Cyanoacrylate Monomer with Acetic Acid

The following composition B was prepared:

| | |
|---|---|
| Methylheptyl cyanoacrylate from Chemence | 97.5 g |
| Glacial acetic acid | 2.5 g |

The following composition C was prepared:

| | |
|---|---|
| DC 1501 Fluid | 40 g |
| DC 245 Fluid | 40 g |
| A composition A | 10 g |
| A composition B | 10 g |

1.5 g of composition C was applied to a lock of 1 g of clean and moist chestnut hair. After a period of 15 minutes, the lock was dried with a hairdryer for 2 minutes.

The lock was colored and the coloration obtained was shampoo-fast. The coloration obtained was more visible than if a composition comprising only the pigment with a low refractive index had been applied.

### Example 5

### Ethylhexyl Cyanoacrylate Monomer

The following composition was prepared:

| | |
|---|---|
| DC 1501 Fluid | 40 g |
| DC 245 Fluid | 40 g |
| A composition A | 10 g |
| Ethylhexyl cyanoacrylate O60 from Tong Shen | 10 g |

1.5 g of the composition was applied to a lock of 1 g of clean and moist chestnut hair. After a period of 15 minutes, the lock was dried with a hairdryer for 2 minutes.

The lock was colored and the coloration obtained was shampoo-fast. The coloration obtained was more visible than if a composition comprising only the pigment with a low refractive index had been applied.

### Example 6

### Butyl Cyanoacrylate Monomer with Acetic Acid

The following composition D was prepared:

| | |
|---|---|
| Butyl cyanoacrylate B-60 from Tong Shen | 90 g |
| Glacial acetic acid | 10 g |

The following composition E was prepared:

| | |
|---|---|
| DC 1501 Fluid | 40 g |
| DC 245 Fluid | 40 g |
| A composition A | 10 g |
| A composition D | 10 g |

1.5 g of the composition E was applied to a lock of 1 g of clean and moist chestnut hair. After a period of 15 minutes, the lock was dried with a hairdryer for 2 minutes.

The lock was colored and the coloration obtained was shampoo-fast. The coloration obtained was more visible than if a composition comprising only the pigment with a low refractive index had been applied.

### Example 7

### Ethoxyethyl Cyanoacrylate Monomer with Acetic Acid

The following composition F was prepared:

| | |
|---|---|
| DC 1501 Fluid | 40 g |
| DC 245 Fluid | 35 g |
| A composition A | 10 g |
| Glacial acetic acid | 5 g |
| Ethoxyethyl cyanoacrylate EO 460 from Tong Shen | 10 g |

1.5 g of the composition F was applied to a lock of 1 g of clean and moist chestnut hair. After a period of 15 minutes, the lock was dried with a hairdryer for 2 minutes.

The lock was colored and the coloration obtained was shampoo-fast. The coloration obtained was more visible than if a composition comprising only the pigment with a low refractive index had been applied.

### Example 8

### Mixture of Methylheptyl Cyanoacrylate and Ethylhexyl Cyanoacrylate Monomers

The following composition H was prepared:

Methylheptyl cyanoacrylate from Chemence 90 g

Ethylhexyl cyanoacrylate 0-60 from Tong Shen 10 g

The following composition I was prepared:

| | |
|---|---|
| DC 1501 Fluid | 40 g |
| DC 245 Fluid | 40 g |
| A composition A | 10 g |
| A composition H | 10 g |

1.5 g of the composition I was applied to a lock of 1 g of clean and moist chestnut hair. After a period of 15 minutes, the lock was dried with a hairdryer for 2 minutes.

The lock was colored and the coloration obtained was shampoo-fast. The coloration obtained was more visible than if a composition comprising only the pigment with a low refractive index had been applied.

### Example 9

### Mixture of Methylheptyl Cyanoacrylate and Butyl Cyanoacrylate Monomers

The following composition J was prepared:

| | |
|---|---|
| Methylheptyl cyanoacrylate from Chemence | 70 g |
| Butyl cyanoacrylate B-60 from Tong Shen | 30 g |

The following composition K was prepared:

| | |
|---|---|
| DC 1501 Fluid | 40 g |
| DC 245 Fluid | 40 g |
| A composition A | 10 g |
| A composition J | 10 g |

1.5 g of the composition K was applied to a lock of 1 g of clean and moist chestnut hair. After a period of 15 minutes, the lock was dried with a hairdryer for 2 minutes.

The lock was colored and the coloration obtained was shampoo-fast. The coloration obtained was more visible than if a composition comprising only the pigment with a low refractive index had been applied.

### Example 10

### Iron Oxide+Organic Pigment

The following composition was prepared:

| | |
|---|---|
| DC 1501 Fluid | 40 g |
| DC 245 Fluid | 40 g |
| A composition L | 10 g |
| Methylheptyl cyanoacrylate from Chemence | 10 g |

0.8 g of the composition was applied to a lock of 1 g of clean and moist chestnut hair. After a period of 15 minutes, the lock was dried with a hairdryer for 2 minutes.

The lock was colored and the coloration obtained was shampoo-fast. The coloration obtained was more visible than if a composition comprising only the pigment with a low refractive index had been applied.

What is claimed is:

1. A dyeing composition comprising, in a suitable dyeing medium, at least one electrophilic monomer, at least one colored pigment and at least one pigment that has a refractive index greater than the at least one colored pigment.

2. A composition according to claim 1, wherein the at least one electrophilic monomer is chosen from at least one monomer of formula (A): 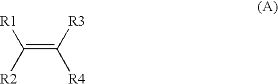
wherein:
R₁ and R₂, which may be identical or different, are each chosen from a group with little or no electron-withdrawing effect, and
R₃ and R₄, which may be identical or different, are each chosen from an electron-withdrawing group.

3. A composition according to claim 2, wherein
R₁ and R₂, which may be identical or different, are each chosen from hydrogen atoms;
saturated or unsaturated, linear, branched or cyclic hydrocarbon-based groups optionally comprising at least one atom chosen from nitrogen, oxygen and sulphur atoms, and optionally substituted with at least one group chosen from —OR, —COOR, —COR, —SH, —SR, —OH, and halogen atoms;
modified or unmodified polyorganosiloxane residues; and
polyoxyalkylene groups,
wherein R is chosen from saturated or unsaturated, linear, branched or cyclic hydrocarbon-based groups comprising from 1 to 20 carbon atoms, optionally comprising at least one atom chosen from nitrogen, oxygen and sulphur atoms, and optionally substituted with at least one group chosen from —OR′, —COOR′, —COR′, —SH, —SR′, —OH, halogen atoms, and a residue of a polymer, wherein R′ is chosen from C₁-C₁₀ alkyl groups.

4. A composition according to claim 2, wherein R₃ and R₄, which may be identical or different, are each chosen from
—N(R)3+,
—S(R)2+,
—SH2+,
—NH3+,
—NO₂,
—SO₂R,
—C≡N,
—COOH,
—COOR,
—COSR,
—CONHR,
—CONH₂,
—F,
—Cl,
—Br,
—I
—OR,
—COR,
—SH,
—SR
—OH,
linear or branched alkenyl groups,
linear or branched alkynyl groups,
mono- or polyfluoro(C₁-C₄)alkyl groups,
aryl groups and
aryloxy groups,
wherein R is chosen from saturated or unsaturated, linear, branched or cyclic hydrocarbon-based groups comprising from 1 to 20 carbon atoms, optionally comprising at least one atom chosen from nitrogen, oxygen and sulphur atoms, and optionally substituted with at least one group chosen from —OR′, —COOR′, —COR′, —SH, —SR′, —OH, halogen atoms, and a residue of a polymer, wherein R′ is chosen from C₁-C₁₀ alkyl radicals.

5. A composition according to claim 1, wherein the at least one electrophilic monomer is chosen from cyanoacrylate monomers of formula (B): 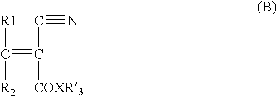
wherein:
X is chosen from NH, S and O,
R′₃ is chosen from hydrogen atoms and R,
R₁ and R₂, which may be identical or different, are each chosen from
hydrogen atoms;
saturated or unsaturated, linear, branched or cyclic hydrocarbon-based groups optionally comprising at least one atom chosen from nitrogen, oxygen and sulphur atoms, and optionally substituted with at least one group chosen from —OR, —COOR, —COR, —SH, —SR, —OH, and halogen atoms;
modified or unmodified polyorganosiloxane residues; and
polyoxyalkylene groups,
wherein R is chosen from saturated or unsaturated, linear, branched or cyclic hydrocarbon-based groups comprising from 1 to 20 carbon atoms, optionally comprising at least one atom chosen from nitrogen, oxygen and sulphur atoms, and optionally substituted with at least one group chosen from —OR′, —COOR′, —COR′, —SH, —SR′, —OH, halogen atoms, and a residue of a polymer, wherein R′ is chosen from C₁-C₁₀ alkyl groups.

6. A composition according to claim 2, wherein R₁ and R₂ are hydrogen atoms.

7. A composition according to claim 1, wherein the at least one electrophilic monomer is chosen from cyanoacrylate monomers of formula (C): 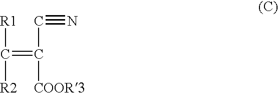
wherein:
R′₃ is chosen from C₁-C₁₀ alkyls and (C₁-C₄)alkoxy(C₁-C₁₀)alkyl radicals,
R₁ and R₂, which may be identical or different, are each chosen from hydrogen atoms;
saturated or unsaturated, linear, branched or cyclic hydrocarbon-based groups optionally comprising at least one atom chosen from nitrogen, oxygen and sulphur atoms, and optionally substituted with at least one group chosen from —OR, —COOR, —COR, —SH, —SR, —OH, and halogen atoms;
modified or unmodified polyorganosiloxane residues; and
polyoxyalkylene groups,
wherein R is chosen from saturated or unsaturated, linear, branched or cyclic hydrocarbon-based groups comprising from 1 to 20 carbon atoms, optionally comprising at least one atom chosen from nitrogen, oxygen and sulphur atoms, and optionally substituted with at least one group chosen from —OR′, —COOR′, —COR′, —SH, —SR′, —OH, halogen atoms, and a residue of a polymer, wherein R′ is chosen from C₁-C₁₀ alkyl groups.

8. A composition according to claim 6, wherein R′₃ is an alkyl radical comprising from 6 to 10 carbon atoms.

9. A composition according to claim 7, wherein R₁ and R₂ are hydrogen.

10. A composition according to claim 1, wherein the at least one electrophilic monomer is an alkyl cyanoacrylate of formula (F): 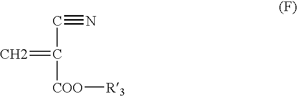
wherein:
R′₃ is chosen from —(CH₂)₇—CH₃, —CH(CH₃)—(CH₂)₅—CH₃, —CH₂—CH(C₂H₅)—(CH₂)₃—CH₃, —(CH₂)₅—CH(CH₃)—CH₃, and —(CH₂)₄—CH(C₂H₅)—CH₃.

11. A composition according to claim 1, wherein the at least one electrophilic monomers is present in an amount ranging from 0.1% to 80% by weight, relative to the total weight of the composition.

12. A composition according to claim 1, wherein the at least one colored pigment and at least one pigment that has a refractive index greater than the at least one colored pigment are in a form chosen from pigmentary pastes and powders.

13. A composition according to claim 12, wherein the at least one colored pigment is chosen from organic pigments.

14. A composition according to claim 13, wherein the at least one organic pigment is chosen from pigments of nitroso, nitro, azo, xanthene, quinoline, anthraquinone and phthalocyanin type, pigments of metal complex type, and pigments of isoindolinone, isoindoline, quinacridone, perinone, perylene, diketopyrrolopyrrole, thioindigo, dioxazine, triphenylmethane and quinophthalone type.

15. A composition according to claim 12, wherein the at least one colored pigment is chosen from composite pigments comprising
at least one particle containing an inorganic core,
at least one organic pigment that at least partially covers the core, and
at least one binder providing attachment of the organic pigments to the core.

16. A composition according to claim 12, wherein the at least one colored pigment is chosen from lakes comprising an inorganic substrate chosen from alumina, silica, calcium sodium borosilicate, calcium aluminium borosilicate, and aluminium, onto which an organic dye is adsorbed.

17. A composition according to claim 12, wherein the at least one colored pigment is a pigment with special effects chosen from pearlescent pigments, pigments with interference effects not bound to a substrate, fluorescent pigments, phosphorescent pigments, photochromic pigments and thermochromic pigments.

18. A composition according to claim 12, wherein the at least one colored pigment is chosen from pearlescent pigments chosen from mica coated with titanium and with iron oxides or with chromium oxide, mica coated with titanium and with an organic pigment, and pearlescent pigments based on bismuth oxychloride.

19. A composition according to claim 1, wherein the at least one pigment that has a refractive index higher than the at least one colored pigment is chosen from mineral pigments.

20. A composition according to claim 19, wherein the mineral pigment is chosen from zirconium oxides, cerium oxides, iron oxides, chromium oxides, manganese violet, ultramarine blue, chromium hydrate, ferric blue and titanium dioxide.

21. A composition according to claim 1, wherein the at least one pigment that has a refractive index higher than the at least one colored pigment is a pearlescent pigment.

22. A composition according to claim 1, wherein the at least one colored pigment and at least one pigment that has a refractive index greater than the at least one colored pigment are each present in an amount ranging from 0.05% to 40% by weight, relative to the total weight of the composition.

23. A composition according to claim 1, wherein the size of the at least one colored pigment and of the at least one pigment that has a refractive index greater than the at least one colored pigment each range from 10 nm to 200 μm.

24. A composition according to claim 1, wherein the composition is anhydrous.

25. A composition according to claim 1, wherein the dyeing medium is chosen from aromatic alcohols, fatty alcohols, modified or unmodified polyols, volatile or non-volatile silicones, mineral, organic or plant oils, oxyethylenated or non-oxyethylenated waxes, paraffins, alkanes, fatty acids, fatty amides and fatty esters.

26. A composition according to claim 1, further comprising at least one nucleophilic agent.

27. A composition according to claim 26, wherein the at least one nucleophilic agent is water.

28. A process for dyeing keratin materials, comprising applying to keratin materials, in the presence of at least one nucleophilic agent, a dyeing composition comprising, in a suitable dyeing medium, at least one electrophilic monomer, at least one colored pigment and at least one pigment that has a refractive index greater than the at least one colored pigment.

29. A process according to claim 28, wherein the dyeing composition further comprises at least one nucleophilic agent.

30. A process according to claim 28, comprising
applying to the keratin materials at least one composition comprising at least one colored pigment and at least one pigment that has a refractive index greater than the at least one colored pigment and
applying to the keratin materials at least one composition comprising at least one electrophilic monomer,
wherein the at least one nucleophilic agent is in the composition comprising the pigments or is in a separate composition.

31. A process according to claim 30, wherein the composition comprising the pigments is an aqueous composition and the composition which comprises the at least one electrophilic monomer is anhydrous.

32. A process according claim 28, wherein the keratin materials are keratin fibers.

33. A dyeing kit comprising:
a first composition comprising at least one pigment,
a second composition comprising at least one electrophilic monomer and
a third composition comprising at least one nucleophilic agent.

34. A kit according to claim 33, wherein the composition comprising the at least one pigment and the composition comprising at least one electrophilic monomer are present in the same anhydrous composition.

35. A process for dyeing keratin materials comprising applying to said materials at least one composition comprising, in a suitable dyeing medium, at least one electrophilic monomer, at least one colored pigment and at least one pigment that has a refractive index greater than the at least one colored pigment.

36. A process according to claim 35, wherein the keratin materials are keratin fibers.
